(19) United States
(12) Patent Application Publication
(10) Pub. No.: US 2024/0374373 A1
(43) Pub. Date: Nov. 14, 2024

(54) PRECLINICAL METRICS TO PREDICT PERFORMANCE OF LENSES

(71) Applicant: AMO GRONINGEN B.V., Groningen (NL)

(72) Inventors: Aixa Alarcon Heredia, Groningen (NL); Hendrik Weeber, Groningen (NL); Antonio del Aguila Carrasco, Groningen (NL); Franck Gounou, Groningen (NL); Srividhya Vilupuru, Groningen (NL); Daniel Black, Groningen (NL); Carmen Cánovas Vidal, Groningen (NL)

(21) Appl. No.: 18/403,604

(22) Filed: Jan. 3, 2024

Related U.S. Application Data
(60) Provisional application No. 63/478,485, filed on Jan. 4, 2023.

(51) Int. Cl. A61F 2/16; A61B 3/00; A61B 3/028

(52) U.S. Cl. CPC A61F 2/1618; A61B 3/0025; A61B 3/028; A61F 2240/002

(57) ABSTRACT

Apparatuses, systems and methods for providing improved ophthalmic lenses, particularly intraocular lenses (IOLs). Examples herein described are directed to predicting a monocular visual acuity (VA) for an intraocular lens. The monocular visual acuity (VA) can be predicted based on one or more metrics calculated from computer simulations or optical bench testing using a set of eyes based on real cataract patients. The monocular visual acuity (VA) can be predicted based on one or more preclinical simulations. Examples herein described are directed to preclinical metrics to predict monocular through focus performance from optical bench data and computer simulations.

### CROSS REFERENCE TO RELATED APPLICATIONS

This application claims the benefit of U.S. Provisional Application No. 63/478,485, filed Jan. 4, 2023, the entire contents of which is hereby incorporated by reference.

### FIELD

The present disclosure relates to lenses, particularly intraocular lenses (IOLs). Apparatuses, systems, and methods may be disclosed herein directed to preclinical metrics for predicting performance of lenses.

### BACKGROUND

Image quality of intraocular lenses (IOLs) when implanted in the eye of a patient may be estimated from different metrics. Many of these metrics are obtained from bench-top optical measurements performed at the best focus of the IOL and at one or a few spatial frequencies and thus can be unreliable in predicting the performance when implanted in the eye and at non-peak focalities. Accordingly, it would be desirable to develop new techniques to reliably predict the performance of an IOL when implanted in an eye.

### BRIEF SUMMARY

Examples herein described may be directed to predicting a monocular visual acuity (VA) for an intraocular lens. The monocular visual acuity (VA) may be predicted based on one or more metrics calculated from computer simulations or optical bench testing using a set of eyes based on real cataract patients. Examples herein described may be directed to predicting a monocular visual acuity (VA) for an intraocular lens based on one or more preclinical simulations.

Apparatuses, systems, and methods may be directed to predicting monocular defocus curves for different intraocular lenses (IOLs) from measurements collected on an optical bench and computer simulations.

In examples, a method for determining intraocular lens performance comprises predicting a monocular visual acuity (VA) for an intraocular lens based on one or more metrics calculated from computer simulations or optical bench testing using a set of eyes based on real cataract patients. The set of eyes may be described by a cornea, a pupil, and an intraocular lens plane. The set of eyes may be further described by a plurality of different intraocular lens models that can be placed in the intraocular lens plane for evaluation. The set of eyes may also be described by a spherocylindrical lens in a spectacle plane to simulate a distance correction for an intraocular lens power. The set of eyes may also comprise a set of eye models that include a corneal geometry of the real cataract patients. The set of eyes may also comprise a set of eye models that include corneal higher order aberrations of the real cataract patients. The set of eyes may also comprise a set of eye models that include axial length of eyes of the real cataract patients. It is further envisioned that the set of eyes may comprise a set of eye models including an intraocular lens simulated in an intraocular lens plane for evaluation.

The one or more metrics may comprise a modulation transfer function or an optical transfer function. The modulation transfer function may comprise an area under the modulation transfer function (MTFa), and the optical transfer function may comprise a weighted optical transfer function (wOTF). The MTFa or the wOTF may be converted into the monocular visual acuity (VA). The conversion from MTFa or the wOTF into the monocular visual acuity (VA) may be calculated based on historical clinical defocus curves or lenses. It is further envisioned that he conversion from MTFa or the wOTF into the monocular visual acuity (VA) may be calculated based on a specific patient population.

The method may include simulating an effect of different levels of defocus and astigmatism on the monocular visual acuity (VA). The method may also comprise calculating a range of vision for the intraocular lens based on the monocular visual acuity (VA). The method may further comprise calculating a tolerance to post-operative refractive errors for the intraocular lens based on the monocular visual acuity (VA).

The method may comprise predicting the monocular visual acuity (VA) for a plurality of different models of intraocular lenses, and determining a mean average defocus curve for the plurality of different models of intraocular lenses. The computer simulations may be performed in white light for an average pupil size. The method may further comprise optimizing a design of the intraocular lens based on the monocular visual acuity (VA). It is also envisioned that the method may comprise selecting the intraocular lens for implantation in an eye of a patient based on the monocular visual acuity (VA).

In examples, a system for evaluating an intraocular lens, the system comprising: one or more processors configured to predict a monocular visual acuity (VA) for the intraocular lens based on one or more metrics calculated from computer simulations or optical bench testing using a set of eyes based on real cataract patients.

The one or more processors may be configured to compare the monocular visual acuity (VA) with a clinical visual acuity. The set of eyes may comprise a set of eye models that include a corneal geometry of the real cataract patients. The set of eyes may comprise a set of eye models including an intraocular lens simulated in an intraocular lens plane for evaluation. The one or more metrics may comprise an area under a modulation transfer function (MTFa), or a weighted optical transfer function (wOTF). The one or more processors may be configured to convert the MTFa or the wOTF into the monocular visual acuity (VA). The one or more processors may be configured to convert the MTFa or the wOTF into the monocular visual acuity (VA) based on historical clinical defocus curves or lenses. The one or more processors may be configured to simulate an effect of different levels of defocus and astigmatism on the monocular visual acuity (VA). The one or more processors may be configured to optimizing a design of the intraocular lens based on the monocular visual acuity (VA). The one or more processors are configured to select the intraocular lens for implantation in an eye of a patient based on the monocular visual acuity (VA).

In examples, a method of optimizing a design of an intraocular lens, the method

comprising: receiving a first lens design for the intraocular lens; predicting a monocular visual acuity (VA) for the first lens design for the intraocular lens based on one or more metrics calculated from computer simulations or optical bench testing using a set of eyes based on real cataract patients; and optimizing the first lens design to a second lens based on the monocular visual acuity (VA).

The method may further comprise comparing the monocular visual acuity (VA) with a clinical visual acuity. It may further comprise optimizing the first lens design to a second lens design based on the comparison of the monocular visual acuity (VA) with the clinical visual acuity at multiple levels of defocus. The method may further comprise comparing a difference between the clinical visual acuity and the monocular visual acuity (VA) with a predetermined tolerance threshold.

The set of eyes may comprise a set of eye models that include a corneal geometry of the real cataract patients. The set of eyes comprise a set of eye models including an intraocular lens simulated in an intraocular lens plane for evaluation. The one or more metrics may comprise an area under a modulation transfer function (MTFa), or a weighted optical transfer function (wOTF).

The method may further comprise converting the MTFa or the wOTF into the monocular visual acuity (VA). It is also envisioned that the method further comprises converting the MTFa or the wOTF into the monocular visual acuity (VA) based on historical clinical defocus curves or lenses. Additionally, the method may further comprise simulating an effect of different levels of defocus and astigmatism on the monocular visual acuity (VA).

### BRIEF DESCRIPTION OF THE DRAWINGS

FIG. 1 illustrates a chart of correlation between a MTFa and a wOTF calculated from a set of eye models and a clinical VA measured in real cataract patients implanted with the same intraocular lens (IOL) models.

FIG. 2 illustrates a chart of RMS error of predicting clinical VA from a MTFa and wOTF from −3.0 D to 0.5 D in steps of 0.5 D of a monofocal lens that slightly extends the depth of focus (sEDF) and a refractive (rEDF) is below 0.05 logMAR.

FIG. 3 provides an average range of vision of different IOL models relative to an aspheric monofocal IOL.

FIG. 4 shows a percentage of eyes in a set of eye models that achieve 0.10 logMAR monocular VA or better under +−0.5 D of defocus and 0.75 D of astigmatism.

FIG. 5 illustrates a system for use in examples herein.

### DETAILED DESCRIPTION

Examples herein described may be directed to predicting a monocular visual acuity (VA) for an intraocular lens. The monocular visual acuity (VA) may be predicted based on one or more metrics calculated from computer simulations or optical bench testing using a set of eyes based on real cataract patients. The monocular visual acuity (VA) may be predicted based on one or more preclinical simulations. Examples herein described may be directed to preclinical metrics to predict monocular through focus performance from optical bench data and computer simulations.

Examples as disclosed herein may be directed to preclinical metrics to predict monocular defocus curves. The monocular defocus curves may be predicted based on optical bench data. Preclinical metrics may be utilized to predict through focus VA from optical bench data.

Preclinical metrics may be useful to determine whether intraocular lens (IOL) designs provide good vision over a range of distances. MTFa and wOTF (as described in U.S. Pat. No. 9,823,163) may be good metrics to predict visual acuity at different distances. The entire disclosure of U.S. Pat. No. 9,823,163 is incorporated by reference herein for all purposes.

MTFa is an area under the MTF (modulation transfer function). The MTFa is the area under the through focus MTF curve captured for multiple spatial frequencies simultaneously (MTFa). The MTFa can be defined using Equation 1 from 0 cycles per mm to 50 cycles per mm, where d determines the sampling size for preclinical measurements at each spatial frequency (f).

$$MTFa = {\sum }_{f = 1}^{50 cpmm / d} \frac{d}{50} MTF ( fd ) \qquad ( Equation 1 )$$

The MTFa can be integrated between 0 cycles per mm and 50 cycles per mm; between 0 cycles per mm and 100 cycles per mm; between 0 cycles per mm and 150 cycles per mm; between 5 cycles per mm and 150 cycles per mm; between 0 cycles per mm and 200 cycles per mm; or there between.

wOTF is a weighted OTF (optical transfer function). The wOTF can be calculated for each defocus position as described in Equation 2 where d determines the sampling size of the spatial frequency (f), PTF is the phase transfer function measured in the optical bench and the CSₜₕ the threshold contrast sensitivity as measured by Green and Campbell (1965) Campbell F W, Green D G. Optical and retinal factors affecting visual resolution. J Physiol. 1965; 181(3): 576-93.

$$wOTF = {\sum }_{f = 1}^{150 cpmm / d} \frac{d}{150} MTF ( fd ) {CS}_{th} ( fd ) cos ( PTF ( fd ) ) \qquad ( Equation 2 )$$

The wOTF can be integrated between 0 cycles per mm and 50 cycles per mm; between 0 cycles per mm and 100 cycles per mm; between 0 cycles per mm and 150 cycles per mm; between 0 cycles per mm and 200 cycles per mm; or there between.

The features of U.S. Pat. No. 9,823,163 may be utilized to predict clinical binocular through focus VA specially from optical bench measurements using an average eye model.

U.S. Pat. No. 8,862,447 proposes a set of eye models, based on data of a real cataract population, to predict VA. The entire disclosure of U.S. Pat. No. 8,862,447 is incorporated by reference herein for all purposes. The method of U.S. Pat. No. 8,862,447 utilized the spatial frequency at which the MTF intersects with the neural threshold function to estimate the visual acuity for a range of defocus positions.

The method proposed herein proposes the use of a set of eye models, based on data from real cataract patients, to calculate the MTFa and the wOTF and to predict the though focus monocular VA. The monocular VA may be predicted for an intraocular lens based on one or more metrics calculated from computer simulations or optical bench testing using a set of eyes based on the real cataract patients.

In examples and methods herein, a set of eye models is created based on data from a group of real cataract patients. Each eye model may be defined by or described by a cornea, a pupil, and an intraocular lens plane. Each eye model may be defined by or described by a corneal geometry. For example, an anterior cornea elevation may be part of the eye model. In combination or alternatively, corneal higher order aberrations of real cataract patients may be utilized. The axial length of eyes of real cataract patients may be included. Different intraocular lens models may be placed in the intraocular lens plane for evaluation. An IOL that is being evaluated may be simulated in the eye model in the intraocular lens plane. The eye models may include an IOL power implanted in the intraocular lens plane. In combination or alternatively, a spherocylindrical lens in a spectacle plane may be provided in the models that may simulate a distance correction for an intraocular lens power (e.g., for optimal refraction). For each eye model, the MTFa and the wOTF are calculated for different levels of defocus.

In the example and methods herein presented, a set of 46 eye models from a normal cataract population was created. The eye models may be those described in “Population-based visual acuity in the presence of defocus well predicted by classical theory” by Henk A. Weeber, Kristen A. Featherstone, and Patricia A. Piers, Journal of Biomedical Optics, Vol. 15(4), July/August 2010, the entire contents of which are incorporated by reference herein for all purposes.

Simulations of the MTFa and the wOTF from −3 D to 0.5 D of defocus in 0.5 D steps were performed in white light using a pupil diameter of 3 mm (e.g., an average pupil size). In an exemplary method, an aspheric monofocal (M), a diffractive EDF (dEDF), and a multifocal (MF) lens were included. Other lenses may be utilized in examples as desired.

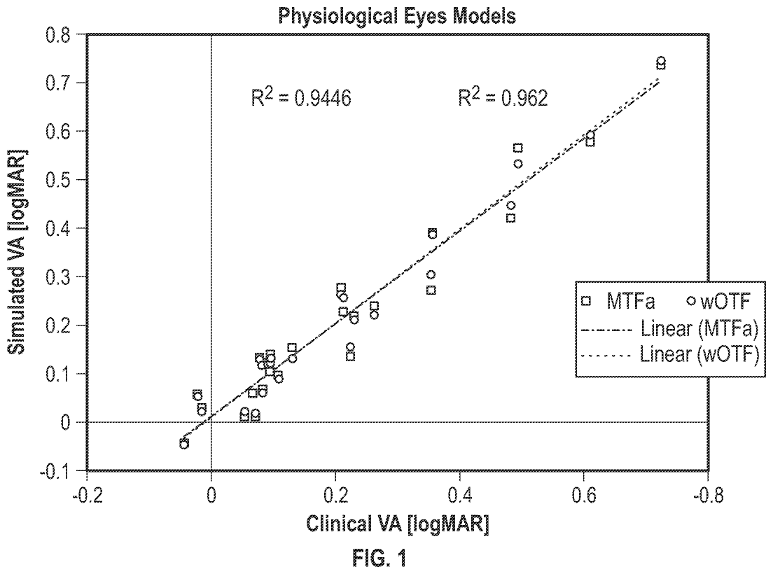

In examples, the calculated metrics (MTFa and the wOTF) may be compared with clinical metrics (clinical VA). FIG. 1, for example, illustrates the good correlation between the MTFa and the wOTF calculated from the set of eye models and the clinical VA measured in real cataract patients implanted with the same IOL models. The correlation is R²=0.94 for the MTFa and 0.96 for the wOTF. The root-mean-square (RMS) error was below 0.05 logMAR for all IOL and metrics except for the aspheric monofocal IOL.

The correlation between the MTFa and/or the wOTF can be used to predict a clinical visual acuity under different levels of defocus for any IOL design. In the example presented herein, the conversion from wOTF and MTFa to the monocular visual acuity (VA) was calculated using historical clinical data (that is, from historical clinical defocus curves or lenses, or the average defocus curves collected for different IOLs in different clinical studies).

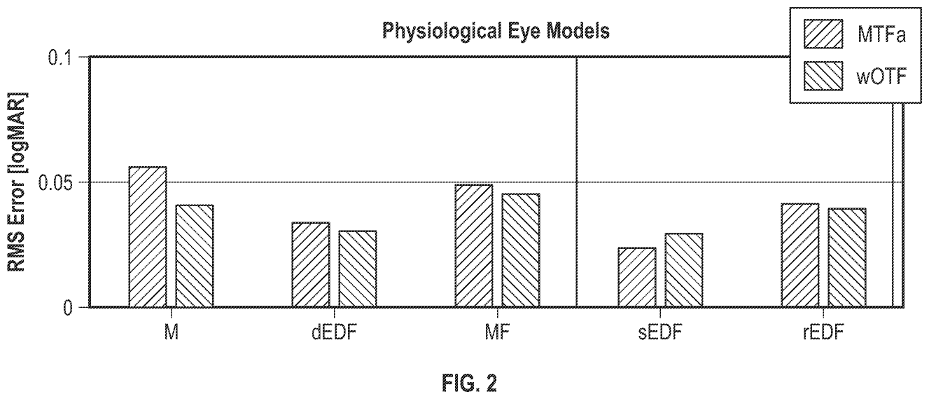

Using this conversion, FIG. 2 shows that the RMS error of the predicting the clinical VA from the MTFa and wOTF from −3.0 D to 0.5 D in steps of 0.5 D of a monofocal lens that slightly extends the depth of focus (sEDF) and a refractive (rEDF) is below 0.05 logMAR. Values for monofocal lenses (M), diffractive extended depth of focus lenses (dEDF) and multifocal lenses (MF) are also shown in FIG. 2.

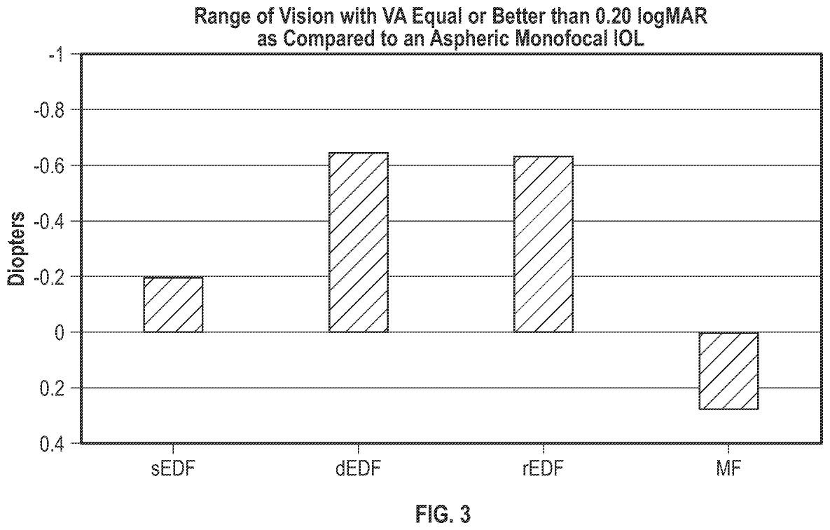

From the simulated VA (monocular visual acuity) provided by the methods presented herein, different preclinical metrics can be used to predict the clinical performance of an intraocular lens. For example, using the correlations found, the mean average defocus curve for the 46 eyes models and for different IOL models (a plurality of different models of intraocular lenses) can be calculated. A range of vision, defined as the range of negative defocus with VA equal or better than 0.20 logMAR can be calculated. A range of vision for one or more IOLs may be calculated based on the simulated VA. FIG. 3 provides the average range of vision of different IOL models (sEDF, dEDF, rEDF, MF) relative to an aspheric monofocal IOL. Negative values mean larger range than the aspheric monofocal IOL.

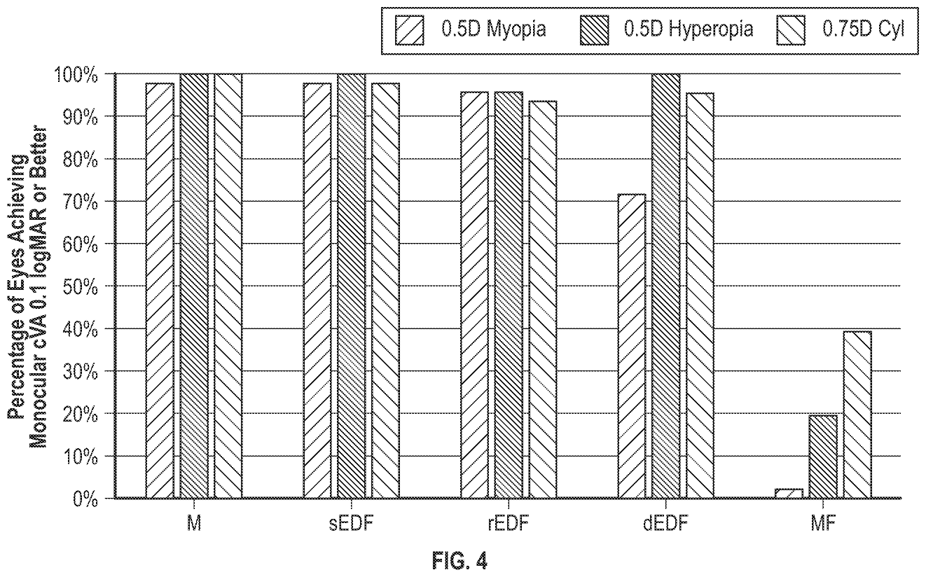

In another example, this methodology can be used to predict the tolerance to post-operative refractive errors of a given IOL design. For example, the correlations between the wOTF calculated using the same set of eye models can be used to calculate the simulated VA under different levels of defocus and astigmatism. From these calculations, the tolerance to post-operative refractive errors of an IOL design can be calculated by estimating, for example, the percentage of eyes that achieve a given VA threshold. For example, FIG. 4 shows the percentage of eyes in the set of eye models that achieve 0.10 logMAR monocular VA or better under +−0.5 D of defocus and 0.75 D of astigmatism.

Simulated VA (sVA) values may be obtained from optical bench and computer simulations for both metrics (MTFa and wOTF), which may be close to clinical data for all lens models, with both metrics yielding comparable results. As such, the methods disclosed herein may be utilized to evaluate a through focus performance of IOLs and the effect of refractive errors on vision.

In computer simulations, a set of eye models based on real cataract populations can be simulated by changing the cornea of the eye model to adjust to different corneal elevations or by inducing corneal aberrations in the entrance pupil.

The set of eye models based on real cataract populations can be modified to evaluate different cataract populations groups and to account for special characteristics of cataract patients. For example, a set of post-LASIK cataract patients can be used to simulate the optical performance of different IOLs in a group of patients that had a LASIK surgery before cataract surgery is performed.

This method can be also applicable to groups of patients with different neural sensitivity, for example glaucoma patients. In that case, the set of eye models can account for different biometry (corneal elevation and axial length) as well as retinal sensitivity (for example, modifying the neural sensitivity function used to calculate the wOTF or finding a different correlation specific for these patients between VA and wOTF/MTFa). In examples herein presented, the conversion from wOTF and MTFa to VA was calculated using historical clinical data (that is, from the average defocus curves of a different cataract populations). However, the conversion can be based on a specific patient population in examples (e.g., glaucoma patients).

Further implementations include selecting an intraocular lens or optimizing a design of an intraocular lens. The selection or optimization may be based on methods disclosed herein, including prediction for a monocular visual acuity (VA).

In examples, if the predicted visual acuity (VA) of an evaluated intraocular lens is below a threshold value, then it may be determined that the evaluated intraocular lens is inappropriate for implantation and thus the evaluated intraocular lens will not be selected for implantation. Physical characteristics of a patient's eye may be measured and it may be determined how the intraocular lens will perform in the patient's eye based on the predicted visual acuity (VA). If the predicted visual acuity (VA) of the evaluated intraocular lens is above the threshold value, then the evaluated intraocular lens may be determined to be appropriate for implantation. Further, the predicted visual acuity (VA) of other models of intraocular lenses may be determined, and if such models have improved visual acuity then such models may instead be selected for implantation in a patient.

In examples, the design of an IOL may be optimized. For example, a system may receive a first lens design for an intraocular lens. A predicted visual acuity (VA) of the first lens design may be provided. The predicted visual acuity may be determined based on the characteristics of a patient's eye that may be measured. If the predicted visual acuity (VA) for the first lens design is below a threshold, then the design may be modified to a second design that may provide sufficient predicted visual acuity (VA). The second design may be determined based on iterative testing of different lens designs, or may be determined based on the calculated predicted visual acuity (VA) of another design that provides the visual acuity (VA) above a threshold. In examples, the first lens design may be modified to a second lens design based on the comparison of the monocular visual acuity (VA) with the clinical visual acuity at multiple levels of defocus. The difference between the clinical visual acuity and the monocular visual acuity (VA) with a predetermined tolerance threshold, for example, may be determined.

### Other Methods may be Utilized Herein.

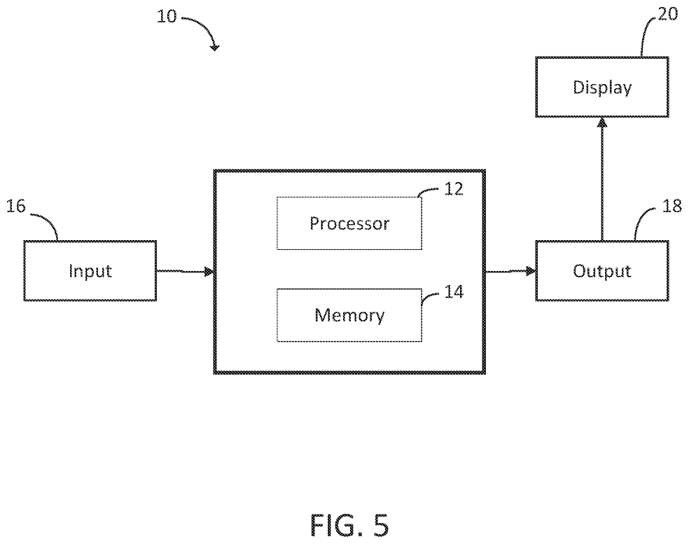

FIG. 5 illustrates a configuration of a system 10 that may be utilized in examples herein. The system 10 may include one or more processors 12 and a memory 14 for storing data and providing such data to the one or more processors 12. The processors 12 may comprise a CPU, microcomputer, or another form of processor (e.g., a distributed network, a cloud computing network, etc.). The memory 14 may comprise non-transitory memory (e.g., a hard disk, RAM, ROM, or other form of memory). An input 16 (e.g., a data terminal or input signal line) and an output 18 (e.g., an output signal line) may be provided. The output 18 may be provided to a display screen 20 or other form of output device (e.g., printer, mobile device, etc.), in examples.

The one or more processors 12 may be configured or programmed to perform any of the methods disclosed herein, including the calculation of the monocular visual acuity (VA), and/or the selection of an intraocular lens or optimization of a design of an intraocular lens, or any other method disclosed herein. The programming of the processors 12 may be stored in the memory 14. The processors 12 may operate based on input provided by the input 16 (e.g., the eye models or characteristics of the IOL being evaluated) and/or stored in the memory 14, and may provide output to the output 18. The processors 12 and system 10 may be embodied in a computer, laptop, tablet, or mobile computer, among other forms of computing devices (e.g., a server or work station) and may comprise a dedicated computing system for performing the methods and processes disclosed herein.

While the present disclosure has been described with respect to various specific examples and embodiments, it is to be understood that the disclosure is not limited thereto and that it can be variously practiced within the scope of the following claims. Combinations of features across various examples may result.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method for determining intraocular lens performance comprising:
predicting a monocular visual acuity (VA) for an intraocular lens based on one or more metrics calculated from computer simulations or optical bench testing using a set of eyes based on real cataract patients.

2. The method of claim 1, wherein the set of eyes are described by a cornea, a pupil, and an intraocular lens plane.

3. The method of claim 2, wherein the set of eyes are described by a plurality of different intraocular lens models that can be placed in the intraocular lens plane for evaluation.

4. The method of any of claims 1-3, wherein the set of eyes are described by a spherocylindrical lens in a spectacle plane to simulate a distance correction for an intraocular lens power.

5. The method of any of claims 1-4, wherein the set of eyes comprise a set of eye models that include a corneal geometry of the real cataract patients.

6. The method of any of claims 1-5, wherein the set of eyes comprise a set of eye models that include corneal higher order aberrations of the real cataract patients.

7. The method of any of claims 1-6, wherein the set of eyes comprise a set of eye models that include axial length of eyes of the real cataract patients.

8. The method of any of claims 1-7, wherein the set of eyes comprise a set of eye models including an intraocular lens simulated in an intraocular lens plane for evaluation.

9. The method of any of claims 1-8, wherein the one or more metrics comprise a modulation transfer function or an optical transfer function.

10. The method of claim 9, wherein the modulation transfer function comprises an area under the modulation transfer function (MTFa), and the optical transfer function comprises a weighted optical transfer function (wOTF).

11. The method of claim 10, wherein the MTFa or the wOTF is converted into the monocular visual acuity (VA).

12. The method of claim 11, wherein the conversion from MTFa or the wOTF into the monocular visual acuity (VA) is calculated based on historical clinical defocus curves or lenses.

13. The method of claim 11, wherein the conversion from MTFa or the wOTF into the monocular visual acuity (VA) is calculated based on a specific patient population.

14. The method of any of claims 1-13, further comprising simulating an effect of different levels of defocus and astigmatism on the monocular visual acuity (VA).

15. The method of any of claims 1-14, further comprising calculating a range of vision for the intraocular lens based on the monocular visual acuity (VA).

16. The method of any of claims 1-15, further comprising calculating a tolerance to post-operative refractive errors for the intraocular lens based on the monocular visual acuity (VA).

17. The method of any of claims 1-16, further comprising predicting the monocular visual acuity (VA) for a plurality of different models of intraocular lenses, and determining a mean average defocus curve for the plurality of different models of intraocular lenses.

18. The method of any of claims 1-17, wherein the computer simulations are performed in white light for an average pupil size.

19. The method of any of claims 1-18, further comprising optimizing a design of the intraocular lens based on the monocular visual acuity (VA).

20. The method of any of claims 1-19, further comprising selecting the intraocular lens for implantation in an eye of a patient based on the monocular visual acuity (VA).

21-40. (canceled)
